(19) United States
(12) Patent Application Publication
(10) Pub. No.: US 2026/0251752 A1
(43) Pub. Date: Aug. 27, 2026

(54) PARTITIONED PROCESSING METHOD FOR RADAR SIGNALS AND RADAR DETECTION DEVICE

(71) Applicant: Wistron Corporation, New Taipei City (TW)

(72) Inventors: Yao Tsung CHANG, New Taipei City (TW); Yin Yu CHEN, New Taipei City (TW); Tsung Yin TSOU, New Taipei City (TW); Kaijen CHENG, New Taipei City (TW)

(21) Appl. No.: 19/213,017

(22) Filed: May 20, 2025

(30) Foreign Application Priority Data
Feb. 24, 2025 (TW) 114106791

(51) Int. Cl. G01S 7/35; A61B 5/05; G01S 7/41; G01S 13/58

(52) U.S. Cl. CPC G01S 7/354; A61B 5/05; G01S 7/415; G01S 13/584

(57) ABSTRACT

A partitioned processing method for radar signals and radar detection device are provided. In the partitioned processing method for radar signals, radar echoes from a field are collected and demodulated through a radar unit to obtain a digital signal in a detection procedure. The digital signal is converted into a range-azimuth matrix. The range-azimuth matrix includes a first region and a second region. A first processing is performed on the first region to obtain first information. A second processing is performed on the second region to obtain second information.

### CROSS-REFERENCE TO RELATED APPLICATION

This non-provisional application claims priority under 35 U.S.C. § 119(a) to Patent Application No. 114106791 filed in Taiwan, R.O.C. on Feb. 24, 2025, the entire contents of which are hereby incorporated by reference.

### BACKGROUND

### Technical Field

The present disclosure relates to a radar technology, and in particular to a partitioned processing method for radar signals and a radar detection device.

### Related Art

At present, radar technology can be used for detecting vital signs and monitor target activities, but they have difference in radar data processing, and it is needed to separately process by multiple radars. If it is processed by the same radar, its required computing power is insufficient to the implementing of two data processing.

### SUMMARY

An embodiment of the present disclosure provides a partitioned processing method for radar signals, which includes: in a detection procedure, collecting and demodulating radar echoes from a field through a radar unit to obtain a digital signal; converting the digital signal into a range-azimuth matrix, where the range-azimuth matrix includes a first region and a second region; performing first processing on the first region to obtain first information; and performing second processing on the second region to obtain second information.

An embodiment of the present disclosure provides a radar detection device, which includes a radar unit and a processing unit. The radar unit is configured to collect and demodulate radar echoes from a field to obtain a digital signal in a detection procedure. The processing unit is configured to convert the digital signal into a range-azimuth matrix, where the range-azimuth matrix includes a first region and a second region; perform first processing on the first region to obtain first information; and perform second processing on the second region to obtain second information.

According to the partitioned processing method for radar signals and the radar detection device provided by some embodiments of the present disclosure, different processing is carried out on different regions in the radar data, so that the data of different detection purposes are obtained for different regions. Therefore, it is not needed to perform multiple times of different processing on the whole range-azimuth matrix, and the time for processing the radar data is decreased. It is not needed to perform different processing separately by multiple radars, and therefore the hardware equipment cost can be reduced.

### BRIEF DESCRIPTION OF THE DRAWINGS

FIG. 1 is a block diagram of a radar detection system according to some embodiments of the present disclosure.

FIG. 2 is a schematic diagram of radar echo signal processing according to some embodiments of the present disclosure.

FIG. 3 is a schematic diagram of digital signals according to some embodiments of the present disclosure.

FIG. 4 is a flowchart of a partitioned processing method for radar signals according to an embodiment of the present disclosure.

FIG. 5A and FIG. 5B are schematic diagrams of a field according to an embodiment of the present disclosure.

FIG. 6 is a flowchart of a partitioned processing method for radar signals according to an embodiment of the present disclosure.

FIG. 7 is a flowchart of a setting procedure before radar signal processing according to an embodiment of the present disclosure.

FIG. 8 is a schematic diagram of time-division multiplexing processing according to an embodiment of the present disclosure.

FIG. 9 is a schematic diagram of time-division multiplexing processing according to an embodiment of the present disclosure.

### DETAILED DESCRIPTION

In order to facilitate the understanding of the technical characteristics, contents and advantages of the present disclosure and the effects it can achieve, the present disclosure is hereby described in detail below in the form of embodiments in conjunction with the accompanying drawings, and the diagrams used therein is only for illustration and supplementary description, and may not be the true proportion and precise configuration after the implementation of the present disclosure. Therefore, the relationship between the proportion and configuration of the accompanying drawings should not be interpreted and the scope of rights in the actual implementation of the present disclosure should not be limited.

In all diagrams, the same reference numeral will be used for representing the same or similar components. The reference to “including” herein is an open term and should be construed as “including, but not limited to”. As used herein, “coupling” means two or more components that are in “direct” physical or electrical contact with each other, or “indirect” with each other in physical or electrical contact.

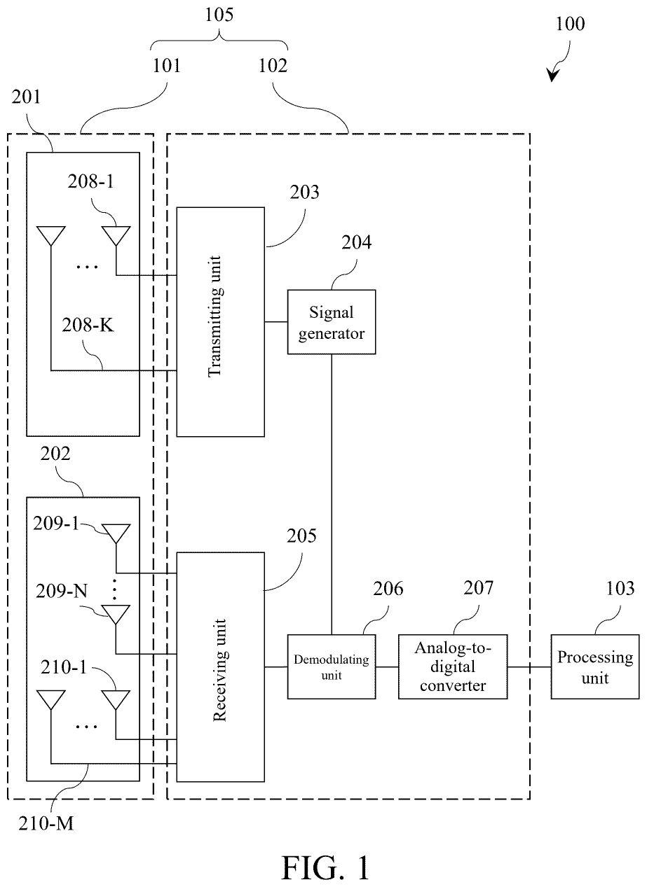

FIG. 1 is a block diagram of a radar detection system 100 according to some embodiments of the present disclosure. With reference to FIG. 1, the radar detection system 100 includes a radar unit 105 and a processing unit 103 which are coupled with each other. The radar unit 105 includes an antenna unit 101 and a front unit 102. The antenna unit 101 is configured to radiate a radio frequency signal to a free space, and the radio frequency signal collides with an object in the free space to reflect a feedback signal. The antenna unit 101 receives the feedback signal (namely radar echo) from the radio frequency signal. The front unit 102 is configured to generate the radio frequency signal, and demodulate and digitize the feedback signal to obtain a digital signal. The processing unit 103 is configured to receive the digital signal and perform signal processing on the digital signal.

In some embodiments of the present disclosure, the radio frequency signal is a Frequency Modulated Continuous Wave (FMCW) signal.

With reference to FIG. 1, the antenna unit 101 further includes a transmitting antenna unit 201 and a receiving antenna unit 202. The transmitting antenna unit 201 includes multiple transmitting antennas 208-1 to 208-K. The transmitting antennas 208-1 to 208-K radiate the radio frequency signal to the free space. The receiving antenna unit 202 includes multiple receiving antennas 209-1 to 209-N and 210-1 to 210-M to receive the feedback signal. K, N and M are positive integers, respectively representing the configuration number of the transmitting antennas 208-1 to 208-K and the receiving antennas 209-1 to 209-N and 210-1 to 210-M. The actual number is determined according to the requirements of the radar detection system 100, which is not limited in the present disclosure. In some embodiments, the receiving antennas 209-1 to 209-N are arranged in an X-axis direction (horizontal direction), and the receiving antennas 210-1 to 210-M are arranged in a Y-axis direction (vertical direction).

The transmitting antennas are generally designed by taking signal transmitting frequency, Field Of View (FOV) and purposes into consideration. The antennas can be designed into a form of lens antennas, patch antennas, or waveguide leaky-wave antennas. In some embodiments of the present disclosure, the transmitting antennas 208-1 to 208-K are the patch antennas.

The receiving antennas are generally designed by taking signal receiving frequency into consideration. In a case of distinguishing the direction of an object, multiple groups of receiving antennas are needed. The receiving antennas receive object echoes from different azimuth angles and accordingly determine the azimuth of the object. The receiving antennas are designed by taking the frequency range of the received radio frequency signal and whether it is needed to distinguish the direction of the object to be detected into consideration. If it is needed to distinguish the direction, the design of Single Input Multiple Output (SIMO) antennas or the design of Multiple Input Multiple Output (MIMO) antennas are needed to be taken into consideration. In some embodiments of the present disclosure, the receiving antennas 209-1 to 209-N and 210-1 to 210-M are the patch antennas and are implemented by a printed circuit board.

As shown in FIG. 1, the front unit 102 includes a signal generator 204, a transmitting unit 203, a receiving unit 205, a demodulating unit 206 and an analog-to-digital converter 207. The signal generator 204 generates the radio frequency signal and simultaneously transmits the radio frequency signal to the transmitting unit 203 and the demodulating unit 206. The transmitting unit 203 includes a power amplifier (PA) for amplifying the radio frequency signal and transmitting the amplified radio frequency signal to the transmitting unit 201 to radiate the radio frequency signal to the free space.

The receiving unit 205 includes a signal amplifier and a filter (not shown in the figure) and is configured to receive the feedback signal received by the antenna unit 101 and amplify and filter the received feedback signal. The demodulating unit 206 is coupled with the signal generator 204 and the receiving unit 205. The demodulating unit 206 receives the radio frequency signal generated by the signal generator 204 and the feedback signal amplified and filtered by the receiving unit 205, demodulates the amplified and filtered feedback signal based on the radio frequency signal, performs frequency mixing and combination, and filters a high-frequency signal. The analog-to-digital converter 207 converts the demodulated feedback signal into the digital signal and transmits the digital signal to the processing unit 103 for subsequent signal processing.

In some embodiments of the present disclosure, the signal generator 204 generates a linear modulation frequency signal with an initial frequency of 77 GHz, an end frequency of 81 GHz and a time cycle Tc of 40 us. In some embodiments of the present disclosure, the signal generator 204 generates a linear modulation frequency signal with the initial frequency of 24 GHz, the end frequency of 28 GHz and the time cycle of 40 us. However, the numeral values of the initial frequency, the end frequency and the time cycle are only examples, the present disclosure is not limited thereto. The position and speed of the object, or the breathing, heartbeat and the like of the object can be detected by using the linear modulation frequency signal and performing proper Fast Fourier Transform (FFT) signal processing. The demodulating unit 206 performs frequency mixing and combination on the modulation frequency signal generated by the signal generator 204 and the feedback signal amplified and filtered by the receiving unit 205, filters the high-frequency signal, and generates an Intermediate Frequency (IF) signal. The analog-to-digital converter 207 converts the IF signal into the digital signal and transmits the digital signal to the processing unit 103 for subsequent signal processing so as to obtain information included in the feedback signal.

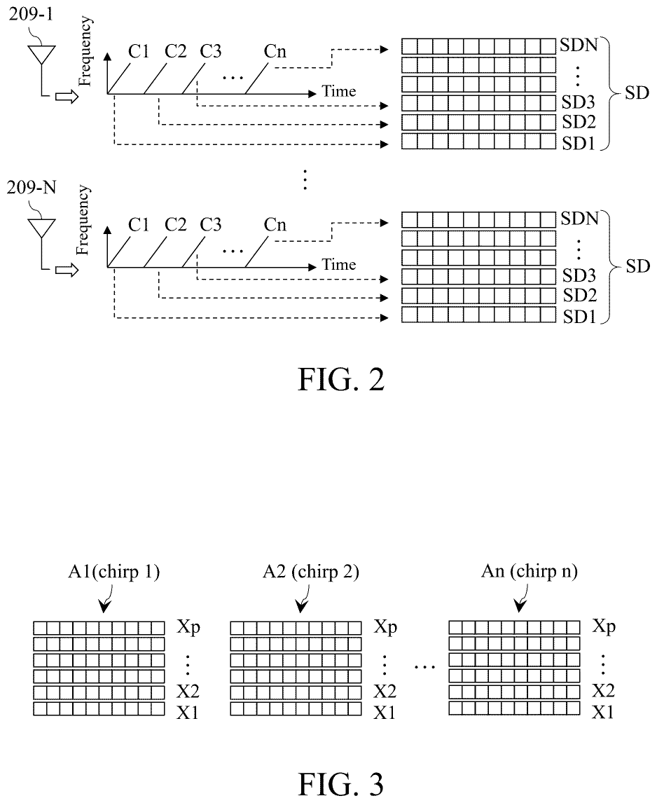

With reference to FIG. 2, FIG. 2 is a schematic diagram of radar echo signal processing according to some embodiments of the present disclosure, and shows that the feedback signals received by the multiple receiving antennas (taking 209-1 to 209-N as an example) in the axial direction are demodulated and converted into the digital signals SD1 to SDN. The feedback signals include multiple chirp signals C1 to Cn in each Frame, and n is a positive integer. The chirp signals C1 to Cn are subjected to linear frequency modulation, and the frequency increases in a linear mode along with time. Each of the chirp signals C1 to Cn is demodulated by the demodulating unit 206 and then converted into the digital signals SD1 to SDN by the analog-to-digital converter 207, and N is a positive integer. That is, the chirp signal C1 is subjected to transmitting, reflecting, receiving, demodulating and analog-to-digital conversion to form the digital signal SD1; the chirp signal C2 is subjected to transmitting, reflecting, receiving, demodulating and analog-to-digital conversion to form the digital signal SD2; and so on. After the chirp signals C1 to Cn of the same frame received by each of the receiving antennas 209-1 to 209-N are converted into the digital signals SD1 to SDN, the chirp signals C1 to Cn can be represented as the digital signals SD1 to SDN in a two-dimensional matrix form shown on the right of FIG. 2.

With reference to FIG. 3, FIG. 3 is a schematic diagram of digital signals SD1 to SDN according to some embodiments of the present disclosure, and shows the other presentation mode of the digital signals SD1 to SDN, the digital signals SD1 to SDN corresponding to the same chirp signal Cx (x ranges from 1 to n, n is a positive integer) received by each of the receiving antennas X1 to Xp (p is a positive integer) in the same frame are arranged into a matrix Ax (x ranges from 1 to n, n is a positive integer). For example, each row of the matrix A1 represents the digital signal SD1 obtained according to the first chirp signal C1 received by each of the receiving antennas X1 to Xp; each row of the matrix A2 is the digital signal SD2 obtained according to the second chirp signal C2 received by each of the receiving antennas X1 to Xp; and so on.

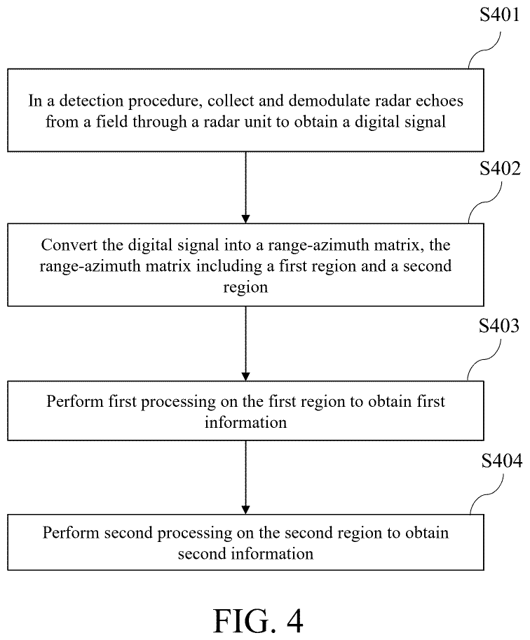

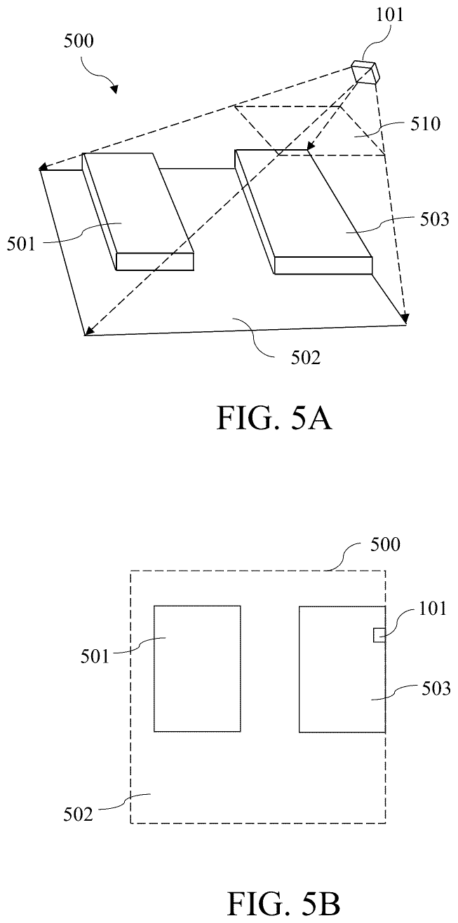

With reference to FIG. 4, FIG. 5A and FIG. 5B. FIG. 4 is a flowchart of a partitioned processing method for radar signals according to an embodiment of the present disclosure. FIG. 5A and FIG. 5B are schematic diagrams of a field 500 according to an embodiment of the present disclosure, FIG. 5A is a three-dimensional schematic diagram of the field 500, and FIG. 5B is a plane schematic diagram of mapping the field 500 in FIG. 5A to a plane 510. The partitioned processing method for radar signals is implemented by the radar detection system 100. After the radar detection system 100 enters the detection procedure, step S401 is performed. The detection procedure is to detect a target in the field 500. The field 500 is partitioned into at least two detection areas. For example, as shown in FIG. 5, the field 500 includes a first detection area 501, a second detection area 502 and a third detection area 503. The third detection area 503 refers to a range out of the first detection area 501 and the second detection area 502 in the field 500. However, the embodiment of the present disclosure is not limited thereto, for example, there may be only two or more than three detection areas.

Step S401 includes: collecting and demodulating radar echoes from the field 500 through the radar unit 105 to obtain the digital signals SD1 to SDN (as shown in FIG. 3).

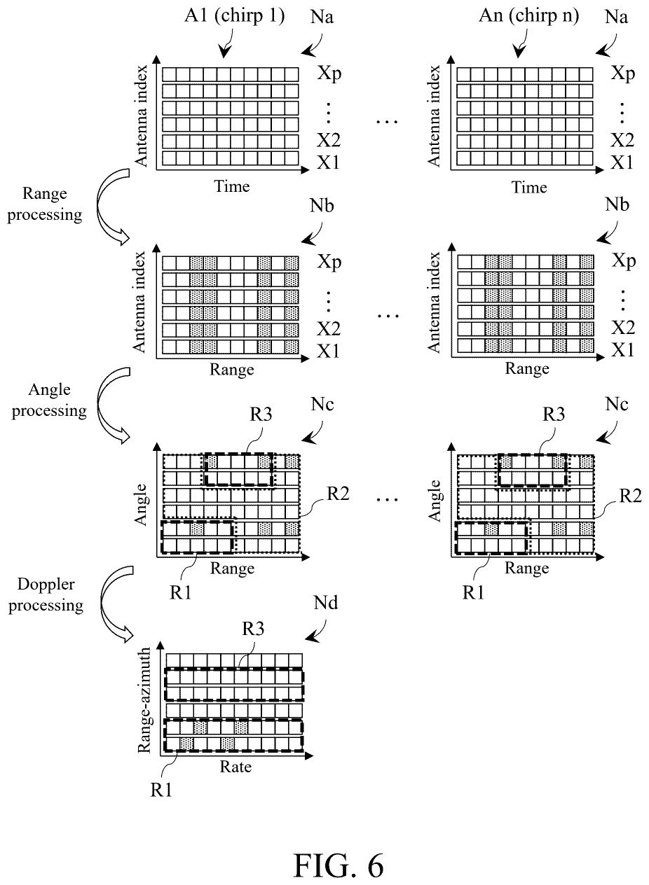

Step S402 includes: converting the digital signals SD1 to SDN into a Range-Azimuth matrix by the processing unit 103. A matrix Nc shown in FIG. 6 is the Range-Azimuth matrix. The matrix Nc includes a first region R1, a second region R2 and a third region R3. The third region R3 refers to the range out of the first region R1 and the second region R2 in the matrix Nc. The Range-Azimuth matrix in this embodiment of the present disclosure includes at least two regions, and when there are only two regions, the second region R2 refers to the range out of the first region R1 in the Nc.

Then, step S403 and step S404 are performed by the processing unit 103, thereby realizing different processing on different regions. For the range-azimuth matrix with two regions, step S403 includes: performing first processing on the first region R1 to obtain first information. Step S404 includes: performing second processing on the second region R2 to obtain second information. In one embodiment, for the range-azimuth matrix with three regions, step S403 further includes: performing the same first processing on the third region R3 to obtain third information. In another embodiment, for the range-azimuth matrix with three regions, step S403 further includes: performing the same second processing on the third region R3 to obtain third information.

Through steps S401 to S404, different processing can be performed on different regions to obtain data of different detection purposes for different regions and reduce the time for processing data. That is, each region is configured to be subjected to only one of the two processing. When a certain region is configured to be subjected to the first processing, the second processing is not performed, and vice versa.

With reference to FIG. 6, FIG. 6 is a flowchart of a partitioned processing method for radar signals according to an embodiment of the present disclosure. Taking processing on a matrix Na as an example, and the matrix Na can be any one of matrixes A1 to An. In some embodiments, step S402 includes: performing location information processing by a beam forming technology. Specifically, the matrix Na is converted into a matrix Nb by range processing, and then the matrix Nb is converted into a matrix Nc by angle processing. The matrix Nc is the range-azimuth matrix.

The range processing includes Range Fast Fourier Transform (Range FFT). In order to detect objects in different ranges (distances), FFT processing is carried out on each of the digital signals SD1 to SDN. The data length of the digital signals SD1 to SDN corresponds to the cycle time of the chirp signals, which can express the information of a fast time. Because the frequency of the chirp signals linearly increases along with time, frequency domain distribution generated after the FFT processing can reflect range distribution (the frequency spectrum is converted into time according to the linear frequency modulation slope, and then the time is converted into range according to the electromagnetic wave transmission speed). Each peak value (such as a color filling block) obtained after FFT processing represents that there is the object at the corresponding range. This method is referred to as Range FFT. The transverse axis of the matrix Nb is the range (distance), and the longitudinal axis of the matrix Nb is an antenna index.

The angle processing includes Angle Fast Fourier Transform (Angle FFT). Because the distance from the object to each antenna is different, the estimation on an Angle of Arrival (AoA) is performed based on phasor change of the peak value of the Range FFT, and it needs at least two receiving antennas 209-1 to 209-N. The direction of the object is detected according to a phase difference between the two antennas. Angle energy distribution is obtained by performing the FFT on a Range bin of the matrix Nb, and the angle is estimated according to the angle peak value, and this method is referred to as the Angle FFT. Each peak value (such as the color filling block) obtained after the Angle FFT processing represents that there is the object at corresponding angle. In some embodiments, the direction angle of the object can be estimated by the Angle of Arrival (AOD) besides the AOA, or other algorithms such as a MUltiple SIgnal Classification (MUSIC) algorithm. The horizontal axis of the matrix Nc represents the range (distance), the longitudinal axis of the matrix Nc represents the angle, namely the range-azimuth matrix, and therefore two-dimensional radar signal distribution is presented. The angle processing can be one-dimensional angle processing (X-axis direction) or two-dimensional angle processing (XY-axis direction) to correspondingly form a two-dimensional matrix Nc or a three-dimensional matrix Nc. In the present disclosure, it is described with the two-dimensional matrix Nc. If the matrix Nc is three-dimensional, the regions in the matrix Nc refer to a three-dimensional space range, and the processing on the regions is correspondingly converted from two-dimensional data processing to three-dimensional data processing.

As shown in FIG. 6, the matrix Nc includes the first region R1, the second region R2 and the third region R3 which respectively correspond to the first detection area 501, the second detection area 502 and the third detection area 503 in FIG. 5A and FIG. 5B. Therefore, different processing can be performed on the radar data of different detection areas in steps S403 and S404.

Taking FIG. 5A and FIG. 5B as examples, the first detection area 501 and the third detection area 503 are static areas (such as beds and sofas), and the second detection area 502 is a dynamic area (such as floors and walkways). The static areas refer to areas in which the target is generally in a static state (such as lying and sitting). The dynamic area refers to an area that the target is generally in a dynamic activity state (such as walking, running and jumping). For the target in the static state, it is suitable for detecting vital sign information (such as breathing, and heartbeat) of the target. For the target in the dynamic activity state, it is suitable for detecting the target activity information (such as activity track, activity state, and activity amount). The activity state refers to walking, running and other activity states. The activity amount refers to accumulated movement amount of a period of time. Therefore, in response to the attribute of a corresponding region refers to the static area (namely the region mapped to the detection area in the field 500 is the static area), the processing unit 103 performs first processing (Doppler processing) in step S403 on the region, and the first information obtained by the first processing is the vital sign information. Relatively, in response to the attribute of the corresponding region refers to the dynamic area (namely the region mapped to the detection area in the field area 500 is the dynamic area), the processing unit 103 performs second processing (point cloud processing) in step S404 on the region, and the second information obtained by the second processing is the target activity information. For example, when the first detection area 501 and the third detection area 503 shown in FIG. 5A and FIG. 5B are the static areas, the first processing is performed on two corresponding regions to obtain the first information and third information which are the vital sign information respectively; and when the second detection area 502 shown in FIG. 5A and FIG. 5B is the dynamic area, the second processing is performed on the corresponding region to obtain the second information that is the target activity information.

As shown in FIG. 6, the Doppler processing is to convert the matrix Nc into a matrix Nd. The Doppler processing includes Doppler Fast Fourier Transform (Doppler FFT). Corresponding peak element of the same position in each matrix Nc is selected to form a one-dimensional array, each element of the one-dimensional array corresponds to ordinal number of each of the chirp signal C1 to Cn, and the information that covers the periods of multiple chirp signals C1 to Cn can express the information of a slow time. The FFT processing is performed on each one-dimensional array to express frequency distribution (frequency offset) of the phase change. The frequency offset is in direct proportion to relative speed of the object and can be converted into a rate. It is referred to as Doppler FFT. Each peak element (such as the color filling block) obtained after Doppler FFT processing represents the cycle change rate (such as life body characteristics like breath, and heartbeat) of the target in a corresponding range azimuth. Therefore, it is only needed to perform Doppler processing on the region needing first processing in the matrix Nc.

The point cloud processing is to perform range processing, Doppler processing, horizontal angle processing and vertical angle processing on the digital signals SD1 to SDN, and then perform coordinate conversion (converting from spherical coordinates into three-axis rectangular coordinates) to obtain the signal intensity of each unit of the rectangular coordinates. According to beam forming data such as the matrix Nc, after points of a static environment and a static object (points in a previous frame) are removed, a point cloud is formed. The point cloud processing may further include: performing clustering analysis on a point cloud map by a clustering algorithm to obtain an object cluster, and computing the center of mass of the object cluster to obtain the location of the object cluster. In some embodiments, the clustering algorithm is based on a density-based spatial clustering of applications with noise (DBSCAN). By tracking the center of mass or the center of gravity of the object cluster, activity information (such as moving speed, moving track, activity state and activity amount) of a corresponding target can be obtained. Therefore, it is only needed to perform point cloud processing on a region needing second processing in the matrix Nc.

In some embodiments, the processing unit 103 further determines an activity behavior of the target according to change in the first information and change in the second information. Specifically, if a certain region has a change in state at a first time and another region has a change in state at a second time, the activity behavior of the target can be determined according to attributes and change sequence of the two detection areas. For one example, if there is a vital sign in the first region R1 which does not have a vital sign originally (namely, there is no peak value in the first region R1 of the matrix Nd originally, but the peak value appears at the first time), and then there is no an activity phenomenon in the second region R2 which has target activity originally (namely, there is the point cloud in the second region R2 of the matrix Nd originally, but the point cloud disappears at the second time), it indicates that the target moves from the second detection area 502 to the first detection area 501 (such as getting on the bed). For another example, if there is no vital signal in the first region R1 which has a vital sign originally (namely, there is peak value in the first region R1 of the matrix Nd originally, but the peak value disappears at the first time), and then there is an activity phenomenon in the second region R2 which has no target activity originally (namely, there is no cloud point originally, but the point cloud appears at the second time), it indicates that the target moves from the first detection area 501 to the second detection area 502 (such as getting off the bed). Through this mode, the activity behavior of the target can be determined as, including but not limited to getting on the bed, getting off the bed, entering a room, getting off the room and the like. The interval between the first time and the second time is determined according to the time required by the activity behavior to be determined.

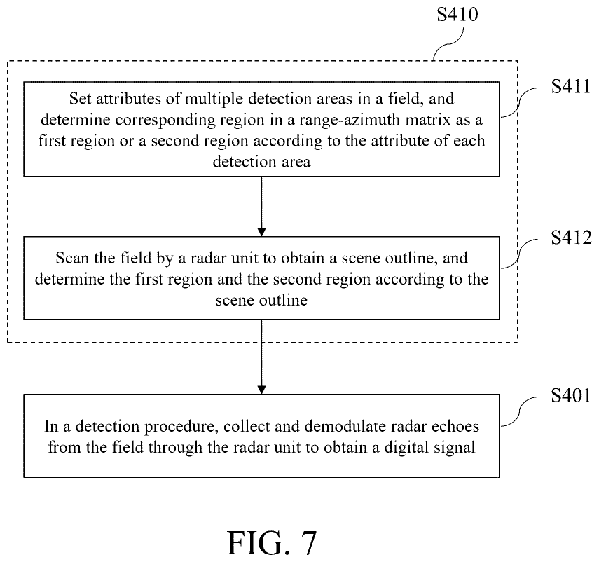

With reference to FIG. 7, it is a flowchart of a setting procedure before radar signal processing according to an embodiment of the present disclosure. In some embodiments, the processing unit 103 further performs the setting procedure before performing the detection procedure (namely before step S401), and the setting procedure includes step S411: setting attributes of multiple detection areas in the field 500, and determining corresponding region as the first region R1 or the second region R2 according to the attribute of each detection area. With reference to Table 1, it shows area setting in this embodiment of the present disclosure. It indicates that the attribute of each detection area can be preset, for example, static area or dynamic area, or the name of a matter placed in the detection area or space name is specifically indicated (it can be classified into the static area or dynamic area). For example, beds, chairs and sofas can be classified into the static areas, and walkways or activity spaces can be classified into the dynamic areas. Furthermore, attributes can also specify detection information contents of the detection area, for example, besides the vital sign information such as breathing and heartbeat or target activity information such as activity amount, it can further include falling detection, sleep work and rest detection, sleep breathing termination detection, off-bed detection or room leaving detection and the like.

TABLE 1

| | Field | Radar | |
|---|---|---|---|
| Detection area | coordinates | coordinates | Attribute |
| First detection | A[X1, Y1], | A′[X1, Y1], | Bed (breathing, |
| area 501 | A[X2, Y2], | A′[X2, Y2], | heartbeat) |
| | A[X3, Y3], | A′[X3, Y3], | |
| | A[X4, Y4], | A′[X4, Y4], | |
| Second detection | B[X1, Y1], | B′[X1, Y1], | Bed (breathing, |
| area 502 | B[X2, Y2], | B′[X2, Y2], | heartbeat, sleep |
| | B[X3, Y3], | B′[X3, Y3], | schedule) |
| | B[X4, Y4], | B′[X4, Y4], | |
| Third detection | C[X1, Y1], | C′[X1, Y1], | Activity Space |
| area 503 | C[X2, Y2], | C′[X2, Y2], | (Activity amount, |
| | C[X3, Y3], | C′[X3, Y3], | fall detection) |
| | C[X4, Y4], | C′[X4, Y4], | |
| | −501-502 | −501-502 | |

As shown in Table 1, the area setting further includes: setting the range of each detection area in the field 500 (which can be defined by four vertex coordinates of a rectangle, namely field coordinates show in Table 1), and setting the corresponding region range of each detection region in the range-azimuth matrix (matrix Nc) (which can be defined by four vertex coordinates of the rectangle, namely radar coordinates shown in Table 1). This embodiment of the present disclosure does not limit the definition mode of the range, for example, it can be defined by more or less polygonal vertex coordinates or other setting modes. In some embodiments, although Table 1 shows two-dimensional coordinates, the present disclosure is not limited thereto, and three-dimensional coordinates (for example, defined by eight vertex coordinates of a cuboid) can also be adopted.

In some embodiments, with reference to FIG. 5B, the location of the antenna unit 101 of the radar projected to the plane 510 from an installation location in the field 500 is used as an origin of the field coordinates, therefore the origin of the field coordinates can be consistent with an origin of the radar coordinates, and it is not needed to perform coordinate conversion processing such as coordinate translation or/and rotation between two coordinates. In some embodiments, it is not to use the location of the antenna unit 101 of the radar as the origin of the field coordinates, but uses other locations as the origin of the field coordinates, and the coordinate conversion processing is further performed between the field coordinates and the radar coordinates.

In Table 1, the third detection area 503 is defined in a mode of deducting the coordinate range of the first detection area 501 and the second detection area 502 from the coordinate range of the field 500.

With reference to FIG. 7, in some embodiments, the processing unit 103 performs the setting program before performing the detection procedure (namely before step S401), and the setting program includes step S412: scanning the field 500 by the radar unit 105 to obtain a scene outline, and determining the first region R1 and the second region R2 according to the scene outline. Specifically, the scene outline can be scanned by location information processing performed by the beam forming technology. Therefore, the arrangement location and arrangement range, the walkway space range and the like of the matter (such as the bed) can be analyzed according to the scene outline. Accordingly, the coordinate ranges of the first detection area 501 and the second detection area 502 can be defined. Moreover, coordinate conversion can be performed according to the coordinate ranges of the first detection area 501 and the second detection area 502, and it is mapped to the range-azimuth matrix (matrix Nc) to obtain the corresponding coordinate ranges of the first region R1 and the second region R2.

In some embodiments, step S411 is not performed, and the region ranges are set according to the coordinate information set by a user in step S412.

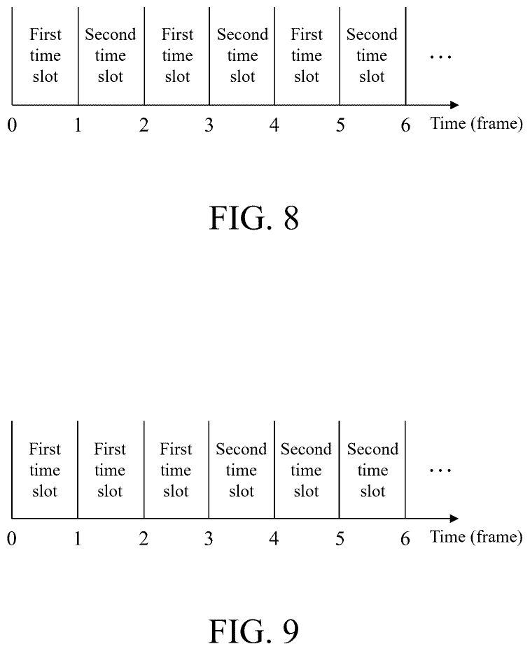

With reference to FIG. 8, it is a schematic diagram of time-division multiplexing processing according to an embodiment of the present disclosure. A time axis includes multiple first time slots and multiple second time slots, the first time slots and the second time slots are arranged in a staggered mode, and the processing unit 103 performs different processing in the period of the two time slots to realize the design of time division multiplexing. For example, the first processing is performed in the period of the first time slots (it can be performed on all the regions needing first processing or only performed on specific regions), the second processing is performed in the period of the second time slots (it can be performed on all the regions needing first processing or only performed on specific regions), and the first processing and the second processing are alternately performed in a time-division multiplexing mode. In some embodiments, the processing unit 103 performs the same processing on different regions in the time-division multiplexing mode. For example, the first processing is performed on the first region R1 in the period of the first time slots, the first processing is performed on the third region R3 in the period of the first time slots, and the second processing is not subjected to the time-division processing. Herein, the period of the first time slots and the period of the second time slots are taken as a frame for exampling, but this embodiment of the present disclosure is not limited thereto.

With reference to FIG. 9, it is a schematic diagram of time-division multiplexing processing according to an embodiment of the present disclosure. Compared with FIG. 8, in this example, the first time slot appears continuously, and the second time slot may also appear continuously.

In some embodiments, the processing unit 103 includes one or more processing modules. In some embodiments, a part of the processing unit 103 is located in the radar unit 105. For example, the processing unit 103 includes a first processing module and a second processing module. The first processing module is located in the radar unit 105 and configured to perform a part of signal processing and transmit a processing result to the second processing module, and the second processing module continues to perform the rest signal processing.

In some embodiments, the processing module includes a processor, an internal memory and a non-volatile memory. The internal memory is a Random Access Memory (RAM) for example. Definitely, the processing module may further include hardware for other functions.

The internal memory and the non-volatile memory are used for storing programs, the programs may include program codes, and the program codes include computer operation instructions. The internal memory and the non-volatile memory provide instructions and data for the processor. The processor reads corresponding computer programs from the non-volatile memory into the internal memory and then runs the computer programs. The processor is specifically configured to perform the steps of the flowchart.

The processor may be an integrated circuit wafer and has signal processing capability. In the implementation process, each method and steps disclosed in the above embodiments can be completed by instructions in a form of an integrated logic circuit of hard or soft in the processor. The processor can be a general processor, including a Central Processing Unit (CPU), a Tensor Processing Unit, a Digital Signal Processor (DSP), an Application Specific Integrated Circuit (ASIC), a Field-Programmable Gate Array (FPGA) or other programmable logic devices, and can implement or execute each method and steps disclosed in the above embodiments.

In some embodiments of the present disclosure, a computer-readable recording medium with stored programs is also provided, the computer-readable recording medium stores at least one instruction, and when the at least one instruction is executed by the processing unit 103, the processing unit 103 can perform each method and steps disclosed in the above embodiments.

Examples of computer-readable recording include, but are not limited to, a phase change memory (PRAM), a static random access memory (SRAM), a dynamic random access memory (DRAM), random access memories (RAM) of other types, a read only memory (ROM), an electrically erasable programmable read only memory (EEPROM), a flash memory or other internal memory technologies, a read only optical disk read only memory (CD-ROM), a digital versatile disk (DVD) or other optical memories, a magnetic tape cassette, a magnetic tape type disk storage or other magnetic storage devices or any other non-transmission media, and can be used for storing information which can be accessed by computing equipment. According to the definition in the present disclosure, the computer-readable medium does not include transitory media, such as modulated data signals and carriers.

According to the partitioned processing method for radar signals and the radar detection device provided by some embodiments of the present disclosure, different processing is carried out on different regions in the radar data, so that the data of different detection purposes are obtained for different regions. Therefore, it is not needed to perform multiple times of different processing on the whole range-azimuth matrix, and the time for processing the radar data is decreased. It is not needed to perform different processing separately by multiple radars, and therefore the hardware equipment cost can be reduced.

What is claimed is:

1. A partitioned processing method for radar signals, comprising:
in a detection procedure, collecting and demodulating radar echoes from a field through a radar unit to obtain a digital signal;
converting the digital signal into a range-azimuth matrix, the range-azimuth matrix comprising a first region and a second region;
performing first processing on the first region to obtain first information; and
performing second processing on the second region to obtain second information.

2. The partitioned processing method for radar signals according to claim 1, wherein the first processing is Doppler processing, and the first information is vital sign information.

3. The partitioned processing method for radar signals according to claim 1, wherein the second processing is point cloud processing, and the second information is target activity information.

4. The partitioned processing method for radar signals according to claim 1, wherein the first processing and the second processing are alternately performed in a time-division multiplexing mode.

5. The partitioned processing method for radar signals according to claim 1, wherein the range-azimuth matrix further comprises a third region; and the partitioned processing method for radar signals further comprises:
performing one of the first processing and the second processing on the third region to obtain third information.

6. The partitioned processing method for radar signals according to claim 5, wherein the processing on the first region and the processing on the third region are alternately performed in a time-division multiplexing mode.

7. The partitioned processing method for radar signals according to claim 1, further comprising:
determining an activity behavior of a target according to change of the first information and change of the second information.

8. The partitioned processing method for radar signals according to claim 1, further comprising:
in a setting procedure, scanning the field by the radar unit to obtain a scene outline; and
determining the first region and the second region according to the scene outline.

9. The partitioned processing method for radar signals according to claim 1, further comprising:
in a setting procedure, setting attributes of a plurality of detection areas in the field; and
determining corresponding region in the range-azimuth matrix as one of the first region and the second region according to the attribute of each detection area.

10. The partitioned processing method for radar signals according to claim 9, wherein in response to the attribute of the region referring to a static area, the first processing is performed on the region, and the first information is the vital sign information; and in response to the attribute of the region referring to a dynamic area, the second processing is performed on the region, and the second information is the target activity information.

11. A radar detection device, comprising:
a radar unit, configured to collect and demodulate radar echoes from a field to obtain a digital signal in a detection procedure; and
a processing unit, configured to:
convert the digital signal into a range-azimuth matrix, the range-azimuth matrix comprising a first region and a second region;
perform first processing on the first region to obtain first information; and
perform second processing on the second region to obtain second information.

12. The radar detection device according to claim 11, wherein the first processing is Doppler processing, and the first information is vital sign information.

13. The radar detection device according to claim 11, wherein the second processing is point cloud processing, and the second information is target activity information.

14. The radar detection device according to claim 11, wherein the first processing and the second processing are alternately performed in a time-division multiplexing mode.

15. The radar detection device according to claim 11, wherein the range-azimuth matrix further comprises a third region; and the processing unit is further configured to perform one of the first processing and the second processing on the third region to obtain third information.

16. The radar detection device according to claim 15, wherein the processing on the first region and the processing on the third region are alternately performed in a time-division multiplexing mode.

17. The radar detection device according to claim 11, wherein the processing unit is further configured to determine an activity behavior of a target according to change of the first information and change of the second information.

18. The radar detection device according to claim 11, wherein the processing unit is further configured to: in a setting procedure, scan the field by the radar unit to obtain a scene outline; and determine the first region and the second region according to the scene outline.

19. The radar detection device according to claim 11, wherein the processing unit is further configured to: in a setting procedure, set attributes of a plurality of detection areas in the field; and determine corresponding region in the range-azimuth matrix as one of the first region and the second region according to the attribute of each detection area.

20. The radar detection device according to claim 19, wherein in response to the attribute of the region referring to a static area, the first processing is performed on the region, and the first information is the vital sign information; and in response to the attribute of the region referring to a dynamic area, the second processing is performed on the region, and the second information is the target activity information.
